(19) United States
(12) Patent Application Publication
(10) Pub. No.: US 2009/0221094 A1
(43) Pub. Date: Sep. 3, 2009

(54) Anthrax Polypeptide Binding

(76) Inventors: M. Amin Arnaout, Chestnut Hill, MA (US); Jian-Ping Xiong, Winchester, MA (US)

Correspondence Address: FISH & RICHARDSON PC, P.O. BOX 1022, MINNEAPOLIS, MN, 55440-1022, US

(21) Appl. No.: 11/909,547

(22) PCT Filed: Mar. 30, 2006
(86) PCT No.: PCT/US06/11762

§ 371 Date: Feb. 10, 2009

Related U.S. Application Data
(60) Provisional application No. 60/666,993, filed on Mar. 31, 2005.

(51) Int. Cl. G01N 33/566; C07K 16/28; C07K 14/705

(52) U.S. Cl. 436/501; 530/389.6; 530/387.9; 530/350

(57) ABSTRACT

Methods are disclosed for detecting anthrax protective antigen polypeptides and inhibiting their binding to β2 integrin α. A domain polypeptides, for example β2 integrin receptors. The disclosed methods are useful, for example, in diagnosing the presence of an anthrax toxin in the environment or in a subject and also for reducing anthrax intoxication in a subject. The methods can also be used to identify compounds that inhibit binding of an anthrax toxin to a β2 integrin.

### TECHNICAL FIELD

This application relates to binding of an anthrax-derived polypeptide to other polypeptides described herein.

### BACKGROUND

Anthrax poses a danger as an agent of biological terrorism. Anthrax is caused by Bacillus anthracis, a gram-positive, spore-forming, rod-shaped bacterium, that unchecked, can cause rampant bacteraemia, multisystem dysfunction, and death. Bacillus anthracis produces three virulence factor proteins: protective antigen (PA), lethal factor (LF), and edema factor (EF). Individually, none of the three proteins is toxic, but a complex of PA and LF, known as lethal toxin, can cause lethal shock. A complex of PA and EF, known as edema toxin, can induce edema. Binding of PA to cell surface receptors allows uptake of lethal toxin or edema toxin, collectively referred to as anthrax toxins.

Two human cellular receptors for PA have been identified recently. These are the cell surface proteins, tumor endothelial marker 8 (TEM8) and capillary morphogenesis protein 2 (CMG2).

### SUMMARY

The methods disclosed are based, in part, on the discovery that a β2 integrin α subunit A domain (β2αA) can bind with high affinity to PA domain 4 (PA-D4).

β2 integrins are heterodimeric receptors, composed of an α subunit and a β subunit. β2 integrins require activation in order to bind physiological ligands. Activation of a β2 integrin induces a high ligand affinity state, termed the open state or open conformation. Activation is normally induced by intracellular signals that travel through transmembrane regions to the extracellular α subunit A domain (β2αA) and β subunit A-like domain (β2βA). Activation can also be initiated by experimental manipulations that mimic physiological stimuli or alternatively by introducing amino acid substitutions in β2αA that “lock” the βαA in an open conformation. As is the case with other β2 integrin ligands, PA-D4 binds to a β2αA when it is in the open conformation.

β2 integrins are expressed on the surface of phagocytes and natural killer cells, which are cells that play a critical role in the immune response. Binding of anthrax toxins to β2 integrins and their subsequent uptake by phagocytes and natural killer cells results in the impaired immune response and intoxication characteristic of anthrax. The interaction of PA-D4 with a β2αA can be exploited to develop methods and identify compounds for inhibiting the binding of anthrax toxins to β2 integrins. Such compounds are putatively useful for reducing the immunosuppression and intoxication associated with anthrax. The high affinity binding of PA-D4 to a β2αA also suggests a means for detecting the presence of PA in a sample (e.g., as a diagnostic assay for detecting infection by B. anthracis and/or exposure of a subject to anthrax toxins).

In one aspect, a high affinity antibody directed against a β2αA (an anti-β2αA antibody) is used to inhibit the binding of a polypeptide that has a PA-D4 (a PA-D4 polypeptide), to a polypeptide that includes a β2αA (a β2αA polypeptide).

In another aspect, the binding of a PA-D4 polypeptide to a β2αA polypeptide is competitively inhibited by the use of a separate and distinct β2αA polypeptide.

In another aspect, that inhibits the binding of PA-D4 polypeptide to a β2αA polypeptide can be identified.

A method for identifying a compound that inhibits the binding of a PA-D4 polypeptide to a β2αA polypeptide, includes determining the affinity of the PA-D4 polypeptide for the β2αA polypeptide, in the presence of a test compound. A decrease in the affinity of the PA-D4 polypeptide for the β2αA polypeptide while in the presence of a test compound indicates that the test compound inhibits binding of PA-D4 polypeptide to the β2αA polypeptide.

Embodiments of the method for identifying an inhibitor of PA-D4 polypeptide to β2αA polypeptide, can include one or more of the following features. For example, the test compound can be a peptide; a peptide that comprises an amino acid sequence from a β2αA; or a peptide that comprises an amino acid sequence from a polypeptide that is a physiological ligand of a β2 integrin receptor. The test compound can be a small molecule, a cell permeable compound, a compound generated through combinatorial synthesis, a compound generated using computer modeling, or a naturally occurring compound purified from a biological source. The β2αA polypeptide, or the PA-D4 polypeptide, or both, can be provided as purified polypeptides. The β2αA polypeptide, the PA-D4 polypeptide, or both, can each further include a transcription factor DNA binding domain (e.g., from GAL-4, LexA, etc.) or a transcription factor transactivation domain (e.g., GAL-4, LexA, VP16, etc.). The β2αA polypeptide, the PA-D4 polypeptide, or both, can each be expressed in a cell. The polypeptides can both be expressed in the same cell. The cell in which a polypeptide to be provided is expressed can be a cell derived from a cell line, a mammalian cell, a yeast cell, or a prokaryotic cell.

In another aspect, a PA-D4 polypeptide can be detected in a test sample. A method to detect the presence of a PA-D4 polypeptide in a test sample includes providing a β2αA polypeptide and contacting it with a detectably labeled ligand (e.g., a labeled PA-D4) that can bind specifically to a β2αA in the presence of the test sample. A decrease in binding of the labeled ligand to β2αA polypeptide in the presence of the test sample indicates the presence of a PA-D4 polypeptide in the test sample.

In another aspect, prophylaxis and treatment for anthrax intoxication in a subject in need thereof includes administering to the subject an antibody that binds specifically to a β2αA and/or an antibody that binds specifically to a β2β A-like domain, such that the antibody inhibits binding of a PA-D4 polypeptide to a β2 integrin receptor expressed in the subject.

In a further aspect, prophylaxis and treatment for anthrax intoxication in a subject includes administering to the subject a β2αA polypeptide such that the β2αA polypeptide competitively inhibits binding of a PA-D4 polypeptide to a β2 integrin receptor expressed in the subject.

Generally, the antibodies or antigen binding portions thereof used in various embodiments bind with an affinity constant of at least 10⁷ M⁻¹, preferably between 10⁸M⁻¹ and 10¹⁰M⁻¹, or about 10⁹M⁻¹. Binding of a PA-D4 polypeptide to a polypeptide that includes a β2αA is inhibited by at least 10%, 15%, 20%, 30%, 40%, 50%, 60%, 70%, 80%, 90% relative to a control.

A PA-D4 can have an amino acid sequence that is at least 90%, 92%, 94%, 96%, 98%, 99%, or 100% identical to that of SEQ ID NO:1 (FIG. 5). A β2αA can have an amino acid sequence that is at least 90%, 92%, 94%, 96%, 98%, 99%, or 1100% identical to that of any one of SEQ ID NOs:2-10.

One or more of the polypeptides provided can be purified polypeptides or alternatively the polypeptides can be expressed by a cell. Cells that can be used to express the polypeptides provided can be cells that are cultured; cells present in an experimental subject (e.g., cells endogenous to a non-human mammal); or cells that are transplanted into an experimental subject. Cells that are used to express the polypeptides provided can be cells that have been genetically manipulated. Cells that are used to express the polypeptides provided can be mammalian cells (e.g., human cells), insect cells, cells derived from a cell line (e.g., a β2 integrin-deficient cell line, a K592 cell line), or prokaryotic cells (e.g., Bacillus anthracis). Cells used to express the polypeptides provided can be subject to treatments known to induce an open conformation of a β2 integrin A domain (e.g., Mn²⁺, phorbol esters, etc.)

In aspects related to the use of an antibody to inhibit the binding of a PA-D4-polypeptide to a β2αA polypeptide, embodiments can include one or more of the following features. Antibodies that compete for binding with a PA-D4 polypeptide, to the β2αA can be antibodies that compete for binding to an epitope recognized by any of the antibodies including: mAb107, KIM127, 7, 44a, CBR, TS1/18, IB4, 24, LM2-1, 60.3, OKM9, OKM1, M1/70, CBRM1 series (anti-CD11b: M1/1, M1/2, M1/4, M1/9, M1/10, M1/13, M1/15, M1/16, M1/17, M1/18, M1/20, M1/21, M1/22, M1/23, M1/24, M1/25, M1/26, M1/27, M1/28, M1/29, M1/30, M1/31, M1/32, M1/33, M1/34) and 904; antibodies used in the method can be antibodies that bind selectively to an conformation state-specific epitope of the β2αA (e.g., a conformational state to which a PA-D4 binds); antibodies used in the method can be humanized antibodies; an antibody that binds specifically to a β2αA can be used together with an antibody that binds specifically to a β2 integrin β subunit A-like domain.

In aspects related to a β2αA polypeptide to inhibit the binding of a PA-D4 polypeptide to another β2αA polypeptide, the inhibiting β2αA polypeptide can include an amino acid sequence that is at least 90% (e.g., 95%, 98%, 99%, or 100%) identical to that of SEQ IDs 2-10; the inhibiting β2αA polypeptide can include an amino acid mutation that causes its A domain to adopt an open, high ligand affinity conformation which permits binding to PA-D4.

In aspects related to the detection of a PA-D4 polypeptide, embodiments can include one or more of the following: the binding affinity of the labeled ligand that binds to the β2αA can be less than the binding affinity of a PA-D4 for the polypeptide comprising a β2αA. The labeled ligand can include a PA-D4 polypeptide; an antibody that binds to the A domain of the polypeptide that includes a β2αA; or a naturally occurring ligand of β2 integrins of which there are at least 30 (e.g., IC3b, ICAM1-4, fibrinogen, NIF, etc.). The labeled ligand or the β2αA polypeptide can include a detectable moiety (e.g., a biological or synthetic fluorophore, an enzyme, enzyme substrate, an antigen, a radiolabel). A detectable moiety can be a quantifiable moiety. The polypeptide that includes a β2αA can be immobilized on a substrate. Enzyme activity can be detected by contacting the enzyme with a reporter that produces a signal (e.g., a flash of light, a color change and the like). A β2αA polypeptide can include a first fluorophore such that binding to a ligand (e.g., PA-D4) labeled with a second fluorophore of causes fluorescence resonance energy transfer between the first fluorophore and the second fluorophore.

In aspects related to prophylaxis and treatment, a subject in need can be any mammal, a non-human mammal, a human, a human in which a PA-D4 polypeptide is present, a human that has been exposed to a microorganism that expresses a PA-D4 polypeptide, e.g., a human that has been exposed to Bacillus anthracis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims

### DESCRIPTION OF DRAWINGS

FIGS. 1A-D are surface plasmon resonance (SPR) sensograms indicating the ability of the indicated polypeptides to interact with PA-D4.

FIG. 2 shows results of a solid phase binding assay of monoclonal antibodies to the purified CD11b/CD18 ectodomain.

FIG. 3 is an SPR sensogram showing the interaction of PA with purified CD11b/CD18 ectodomain and the lack of interaction in the presence of the monoclonal antibody mAb 107.

FIG. 4 shows results of a cell viability assay in the presence of a PA-diphtheria toxin (1×10⁻¹⁰ M) with increasing concentrations of recombinant Von Willebrand type A domain from ATR, purified from either a mammalian cell line or E. coli, added to the cell culture.

FIG. 5 lists the amino acid sequence of Anthrax Protective Antigen Domain 4 (SEQ ID NO:1).

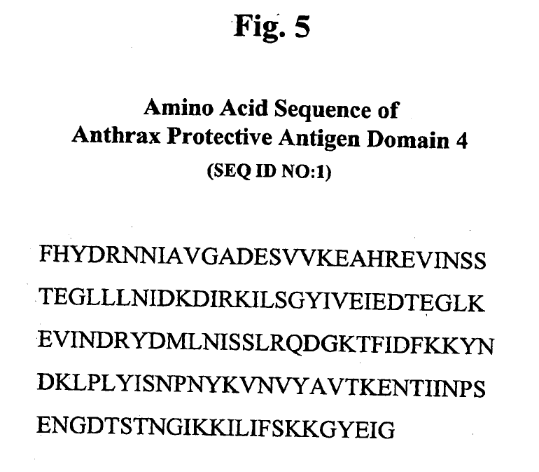

FIG. 6 lists the amino acid sequence of the Human CD11b A domain (SEQ ID NO:2). A structurally conserved isoleucine critical to activation is highlighted (bold, underline, and asterisk).

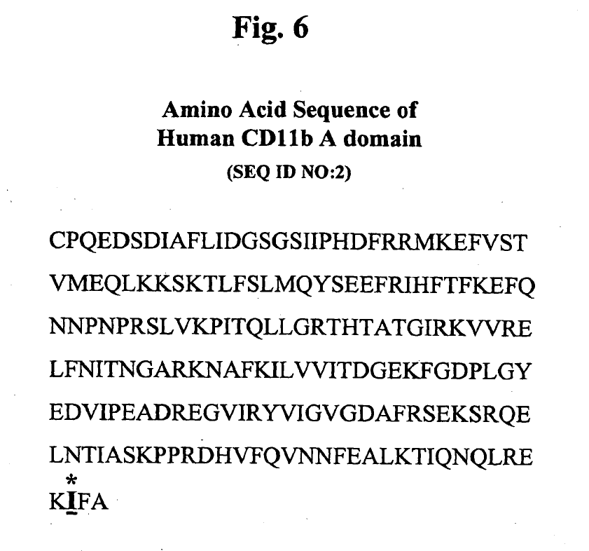

FIG. 7 lists the amino acid sequence of the Human CD11c A Domain (SEQ ID NO:3). A structurally conserved isoleucine critical to activation is highlighted (bold, underline, and asterisk).

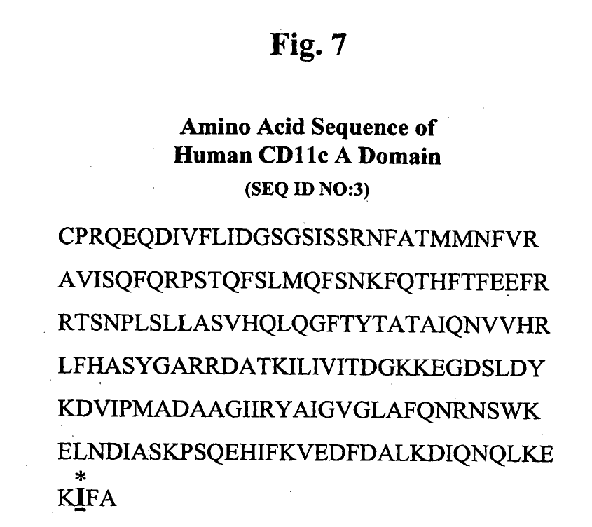

FIG. 8 lists the amino acid sequence of the Human CD11d A Domain (SEQ ID NO:4). A structurally conserved isoleucine critical to activation is highlighted (bold, underline, and asterisk).

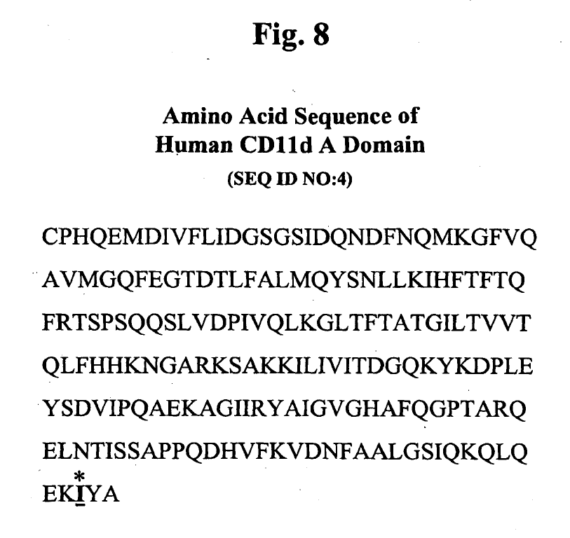

FIG. 9 lists the amino acid sequence of the Human CD11a A domain (SEQ ID NO:5). A structurally conserved isoleucine critical to activation is highlighted (bold, underline, and asterisk).

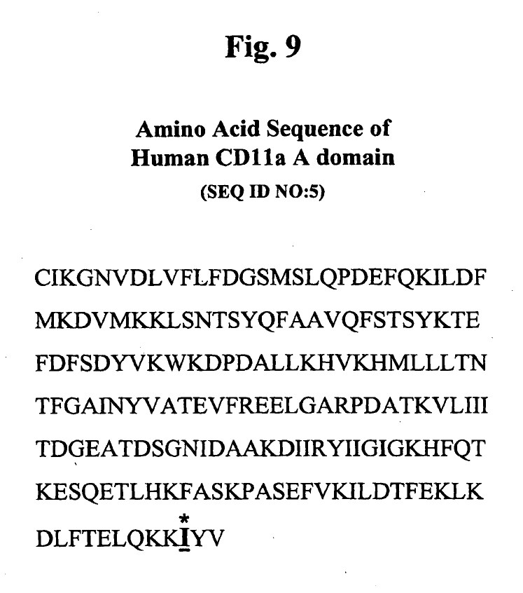

FIG. 10 lists the amino acid sequence of the Human Alpha 1 (CD49a) A domain (SEQ ID NO:6). A structurally conserved isoleucine critical to activation is highlighted (bold, underline, and asterisk).

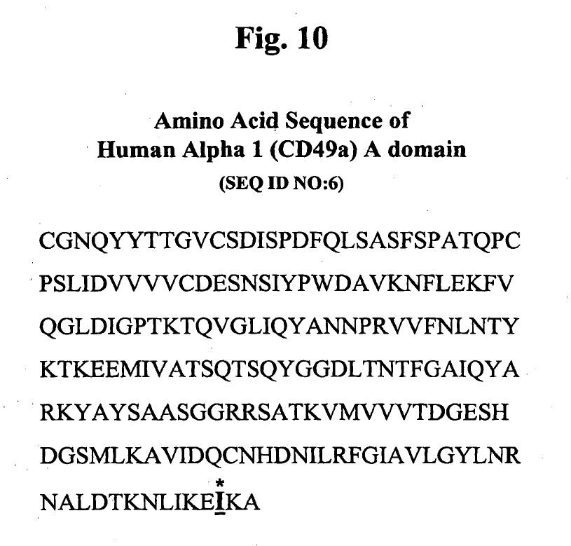

FIG. 11 lists the amino acid sequence of the Human Alpha 2 (CD49b) A domain (SEQ ID NO:7). A structurally conserved isoleucine critical to activation is highlighted (bold, underline, and asterisk).

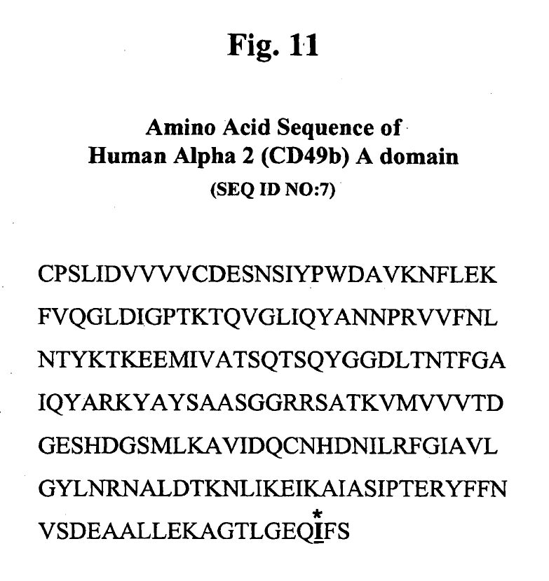

FIG. 12 lists the amino acid sequence of the Amino Acid Sequence of Human Alpha 10 A domain (SEQ ID NO:8). A structurally conserved isoleucine critical to activation is highlighted (bold, underline, and asterisk).

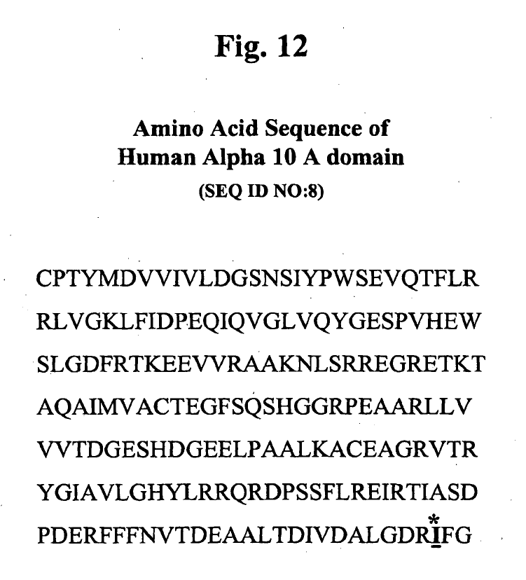

FIG. 13 lists the amino acid sequence of the Human Alpha 11 A domain (SEQ ID NO:9). A structurally conserved isoleucine critical to activation is highlighted (bold, underline, and asterisk).

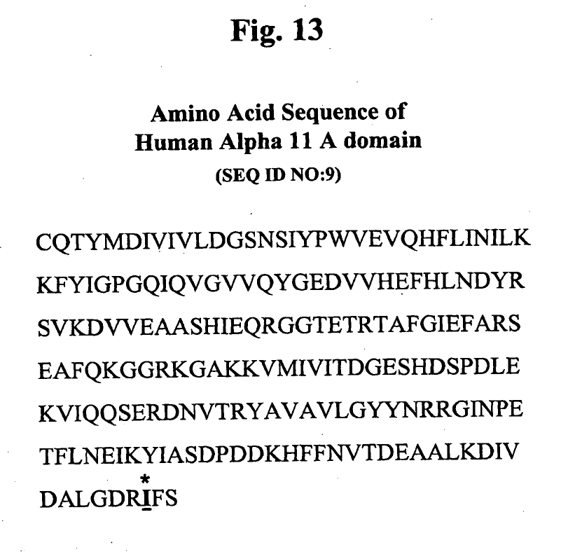

FIG. 14 lists the amino acid sequence of the Human Alpha E A domain (SEQ ID NO:10). A structurally conserved isoleucine critical to activation is highlighted (bold, underline, and asterisk).

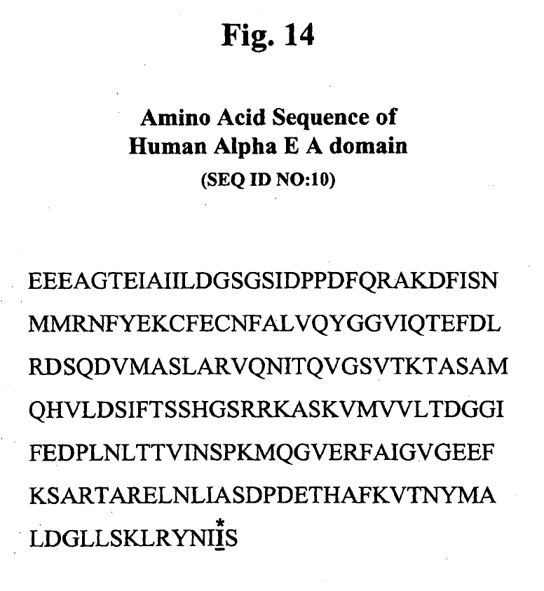

FIG. 15 lists the amino acid sequence of the Human CD18 A-like Domain. A structurally conserved leucine critical to activation is highlighted (bold, underline, and asterisk). (SEQ ID NO:11).

### DETAILED DESCRIPTION

Methods are disclosed herein which are based, in part, on the discovery that anthrax PA-D4 can bind to an integrin β2αA domain when it is in an open (high affinity) conformation. The methods disclosed herein are useful, for example, in the detection of an anthrax toxin (i.e., one that includes the anthrax protective antigen) in, e.g., an environmental sample (e.g., a water supply), or a biological sample (e.g., a blood sample). Methods are also disclosed for reducing binding of an anthrax toxin to a cell where the binding is mediated by PA binding to a β2αA target. Such methods can be useful in reducing anthrax intoxication, e.g., during a bioterrorist attack with anthrax spores or toxins. Methods for inhibiting are based on competitive blocking of PA-D4 binding to β2αA by an antibody or another β2αA polypeptide to which the PA-D4 can bind or by a small molecule. The methods are also useful for screening compounds that can disrupt the interaction of PA-D4 with β2αA. The methods are described followed by a more detailed description of the polypeptides and antibodies used in the methods.

### I. Assays for PA-D4 to β2αA Binding In Vitro

In some embodiments, binding of PA-D4 to β2αA is assayed in a cell free binding assay utilizing fully or partially purified polypeptides or cell extracts. The PA-D4 to β2αA assay can be used, e.g., to identify compounds that effectively inhibit the interaction of PA-D4 with β2αA.

Purified polypeptides include polypeptides that are generated in vitro (e.g., by in vitro translation or by use of an automated polypeptide synthesizer) and polypeptides that are initially expressed in a cell (e.g., a prokaryotic cell, a eukaryotic cell, an insect cell, a yeast cell, a mammalian cell, a plant cell) and subsequently purified. Embodiments of cells expressing one of the polypeptides described above include, for example, cells that express a polypeptide encoded by an endogenous gene, cells transduced with an expression vector encoding a polypeptide, and cells that are experimentally manipulated to induce expression of an endogenous gene that is not typically expressed in that cell type (e.g., gene activation technology). In some embodiments, polypeptides are fusion proteins (e.g., a β2αA-glutathione-S-transferase fusion) that may include a protease cleavage site to allow cleavage and separation of the fusion protein into separate polypeptides. In some embodiments, a polypeptide can include an amino acid sequence that facilitates purification of the polypeptide (e.g., a multiple histidine tag, a FLAG tag, etc). Methods for isolating proteins from cells or polypeptides that are expressed by cells, include affinity purification, size exclusion chromatography, high performance liquid chromatography, and other chromatographic purification methods. The polypeptides can be post-translationally modified, e.g., glycosylated. In some embodiments, the β2αA polypeptide includes an open form (high ligand affinity) of the β2αA (e.g., a CD11B A domain with an 1333A mutation).

Soluble and/or membrane-bound forms of purified polypeptides can be used in the cell-free assays disclosed. When membrane-bound forms of a protein are used (e.g. a β2 integrin receptor), it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In some embodiments, using a cell free binding assay, binding of a PA-D4 to a β2αA polypeptide can be quantified using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules. The presence of a compound that inhibits the binding of a PA-D4 to a β2αA, results in a proportional decrease in a BIA signal. The concentration of each of the polypeptides tested for binding is between at least 10 nM and 500 μM and can be carried out in the presence of a divalent cation (e.g., Ca²⁺, Mg²⁺, or Mn²⁺) at a divalent cation concentration of about 2 mM.

In other embodiments, interaction between two molecules can also be detected, e.g., using fluorescence resonance energy transfer (FRET) (see, for example, Lakowicz et al., U.S. Pat. No. 5,631,169; Stavrianopoulos et al., U.S. Pat. No. 4,868,103). A fluorophore label on the ‘donor’ molecule is selected such that its emitted fluorescence will excite a fluorophore on an, ‘acceptor’ molecule for which emission is the FRET signal. Alternatively, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection (e.g., using a fluorimeter). The presence of an inhibitor of PA-D4 to β2αA binding, results in a proportional decrease in a FRET (i.e., a reduced acceptor fluorescence emission).

The β2αA or PA-D4 polypeptides can anchored onto a solid phase. PA-D4-β2αA complexes that are anchored on the solid phase can be detected at the end of the reaction. In some embodiments, a polypeptide can be anchored onto a solid surface, and an inhibitor of binding which is not anchored can be labeled, either directly or indirectly, with a detectable label.

It may be desirable to immobilize either PA-D4 or β2αA polypeptides to facilitate separation of complexed from uncomplexed forms of one or both of the proteins, as well as to accommodate automation of the assay. Binding of a PA-D4 polypeptide to a β2αA polypeptide can be accomplished in any vessel suitable for containing the reactants. Examples of such vessels include microtiter plates, test tubes, and micro-centrifuge tubes. In some embodiments, a fusion protein can be provided which adds a domain that allows one or both of the proteins to be bound to a matrix. For example, glutathione-S-transferase fusion protein that has a PA-D4 or a β2αA, can be adsorbed onto glutathione sepharose beads (Sigma Chemical, St. Louis, Mo.) or glutathione derivatized microtiter plates, which are then combined with a binding inhibitor and either the non-adsorbed PA-D4 or β2αA polypeptide, and the mixture is incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of binding between the β2αA and the PA-D4 is determined using standard techniques.

Other techniques for immobilizing the polypeptides on matrices, include using conjugation of biotin and streptavidin. Biotinylated polypeptides can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized components are added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the bound component (the antibody, in turn, can be directly labeled or indirectly labeled with, e.g., a labeled anti-Ig antibody).

In some embodiments, this assay can be performed utilizing antibodies reactive with a PA-D4 or β2αA polypeptide, but which do not interfere with PA-D4-β2αA binding. Such antibodies can be linked to the wells of the plate, and unbound PA-D4 or β2αA polypeptide can in turn be linked to the wells by antibody binding. Methods for detecting such complexes, in addition to those described above for the GST-immobilized complexes, include immunodetection of complexes using antibodies reactive with the PA-D4 or β2αA polypeptides, as well as enzyme-linked assays which rely on detecting an enzymatic activity associated with the presence of PA-D4 or β2αA polypeptides.

In some embodiments, cell free assays can be conducted in a liquid phase. In such an assay, the reaction products are separated from unreacted components, by any of a number of standard techniques, including chromatography, electrophoresis and immunoprecipitation.

### II. Inhibition of PA-D4 to β2αA Binding In Vitro

In Vitro inhibition of binding of PA-D4 to β2αA binding can be implemented by contacting a purified β2αA polypeptide with one or more of antibodies against a β2 integrin α subunit A generated (e.g, those described below). Antibodies against β2αA competitively inhibit binding of PA-D4 to β2αA.

The antibody can be directed against an epitope found in an open form of a β2αA (e.g., the mAb 107 antibody described below). In some embodiments, the β2αA is part of a complex with a β2β A like domain (e.g., a β2 integrin receptor) to form a heterodimer. Binding of PA-D4 to the heterodimer can be inhibited by contacting the heterodimer with an antibody against the β2αA, and in addition, an antibody against the β2βA-like domain.

PA-D4 to β2αA binding can be competitively inhibited by a first polypeptide by contacting the PA-D4 with another β2αA polypeptide. The competitor β2αA polypeptide can have an open form (high ligand affinity) form the A domain. In some embodiments, the competitor β2αA polypeptide is in stoichiometric excess relative to the PA-D4 polypeptide.

### III. Assays of PA-D4 to β2αA Binding Ex Vivo

In some embodiments of the disclosed methods, one or more of the polypeptides is expressed in a cell. For example, β2αA polypeptide-expressing cell can be contacted with a purified PA-D4 polypeptide. In various embodiments, cells that can be used include cells that express a polypeptide encoded by an endogenous gene, cells transduced with an expression vector encoding a polypeptide, and cells that are experimentally manipulated to induce expression of an endogenous gene that is not typically expressed in that cell type (e.g., gene activation technology). The cells that β2αA polypeptide can be cultured eukaryotic cells, e.g., a mammalian cell line, a human cell line, or a cell deficient in endogenous β2 integrin expression (e.g., a K596 or K562 cell line).

In some embodiments, the polypeptide expressed by a cell in culture is part of a β2 integrin receptor (e.g., a CD11B/CD18 receptor expressed on the surface of a macrophage).

Methods for introducing expression vectors into cells, stably or transiently include all standard methods known to those of ordinary skill in the art.

In some embodiments, cells expressing a β2αA polypeptide are first exposed to a treatment known to activate β2 integrins, for example incubation with Mn²⁺ or phorbol myristate acetate, prior to or during contact with a PA-D4 polypeptide.

A number of assays can be used to determine PA-D4 to β2αA polypeptide binding, when the β2αA polypeptide is expressed on the surface of a cell. In some embodiments, the PA-D4 polypeptide is fluorescently labeled. Fluorescent labeling can be achieved by generating a fusion protein that includes a biofluorescent polypeptide. Suitable biofluorescent polypeptides include enhanced green fluorescent protein (EGFP) and well known variants of EGFP such as EYFP as well as other fluorescent proteins such as DS-Red. The PA-D4 polypeptide can be labeled chemically with a suitable fluorophore. Examples of suitable fluorophores include, fluorescein isothiocyanate, rhodamine, Texas Red, Oregon Green, Alexa™ dyes, etc.

In some embodiments, binding of a PA-D4 polypeptide to a β2αA-expressing cell is done in a physiological buffer (e.g., phosphate buffered saline) containing divalent cations (e.g., 2 mM Mn²⁺), on ice. The labeled PA-D4 polypeptide can be added to the cells at a concentration of between 0.01 μM and 10 μM, for between 0.5 to 4 hours (e.g., for two hours). After incubation of the cultured cells with the fluorescently labeled polypeptide, excess polypeptide is washed out and bound fluorescently labeled polypeptide can be quantified by standard methods for measuring fluorescence in cells (e.g., flow cytometric analysis). Methods for quantifying fluorescence include a method for determining background fluorescence (e.g., the amount of PA-D4-associated fluorescence detected on the surface of cells that do not express a β2αA). Inhibition of PA-D4 binding is demonstrated by a decrease in the amount of cell surface-bound fluorescence.

The PA-D4 can be labeled by non-fluorescent means, for example by biotinylation, or radiolabeling (e.g., ¹²⁵I iodination). Bound PA-D4 polypeptide can be determined by standard methods for detecting biotin (e.g., using an avidin conjugated enzyme) or for detecting radioactivity (e.g., scintillation counting), respectively.

In other embodiments, cells expressing a β2αA polypeptide on their surface are incubated with (1) a PA-D4 fusion polypeptide that includes the lethal factor (LF) binding domain of anthrax protective antigen, (2) a fusion protein that includes the PA binding domain of LF fused to a toxin protein, for example the diphtheria toxin catalytic A chain, each at a concentration of approximately between 10⁻¹¹ and 10⁻⁸ M. After incubating cells with these components for approximately 24 hours, cell viability is assessed by any standard cell viability assay. Cell viability will be inversely correlated with the amount of cell surface-bound polypeptide having an anthrax protective antigen domain, therefore inhibition of PA-D4 binding will be indicated by a relative increase in cell viability in this assay.

In other embodiments, the PA-D4 polypeptide (e.g., anthrax protective antigen) is expressed and secreted by a cell. The cell can be a cultured prokaryotic cell (e.g., B. anthracis) that secretes the PA-D4 polypeptide into growth medium. The growth medium that contains the secreted polypeptide can be used to contact cultured cells, preferably cultured mammalian cells that express a β2αA on the their surface. In other embodiments, the PA-D4 polypeptide can be expressed and be secreted by the same type of cultured cell (e.g., a mammalian cell) that expresses a β2αA on its surface. In this embodiment the cells expressing the PA-D4 polypeptide and the cells expressing a β2αA polypeptide can be co-cultured, so that the PA-D4 polypeptide from one population of cells is secreted into the medium that is shared with another population of cells expressing a β2αA on their cell surface. In other embodiments, small molecules (M.W. less than 500 Daltons) are screening to identify inhibitors of the interaction between a PA-D4 polypeptide to a β2αA polypeptide.

### IV. Inhibition of PA-D4 to β2αA Binding, Ex Vivo

In some embodiments, one or more of the antibodies (described below) that bind to a β2αA can be used to inhibit binding of PA-D4 to a β2αA expressed on the surface of a cell. In some embodiments, cultured, β2αA-expressing cells are contacted with one or more antibodies to the β2αA prior to contacting the cells with the PA-D4 polypeptide. In other embodiments, the β2αA antibodies can be added at various times after adding the PA-D4 polypeptide (e.g., 5 minutes after, 10 minutes after, 30 minutes after, etc). In some embodiments, cells are treated to induce activation of a β2αA (e.g., by contacting the cells with a phorbol ester). In some embodiments, antibodies bind specifically to an open form of a β2αA. In some embodiments it may be useful to add a protease inhibitor to prevent degradation of exogenous polypeptides and/or antibodies used to contact cultured cells.

In some embodiments, the cells are contacted with a combination of β2αA and β2αA-like domain antibodies. Antibodies can be added to cells at a concentration of about 0.004 nM to 400 nM. The concentration of antibodies added is commonly in direct proportion to the concentration of PA-D4 polypeptide added to the cells.

In some embodiments, β2αA polypeptides are used to competitively inhibit the binding of a PA-D4 polypeptide to a β2αA polypeptide expressed by cells in culture. The competing β2αA polypeptide can be added to the cells before or after adding PA-D4 polypeptides. Polypeptide binding is done under the same conditions as described above for Ex Vivo inhibition of polypeptide binding using antibodies. The purified competing β2αA polypeptide can be added at a concentration that is at least equal to the concentration of added PA-D4 polypeptide. The competing β2αA polypeptide can have at least one mutation that stabilizes the open, high ligand affinity, form of the A domain.

In some embodiments, two populations of cells (e.g., derived from a K592 cell line) can be co-cultivated. A first population of cells expresses a β2αA polypeptide on the cell surface and a second population of cells expresses a β2αA polypeptide that is secreted into the medium shared by the two populations of cells. In other embodiments, small molecules (M.W. less than 500 Daltons) are screening to identify inhibitors of the interaction between a PA-D4 polypeptide to a β2αA polypeptide.

### V. Inhibition of PA-D4 to β2αA Binding In Vivo

In some embodiments, PA-D4 to β2αA binding is inhibited in vivo, for exampled in an experimental subject (e.g., a rat, a mouse, a rabbit, or a monkey). In some embodiments a purified PA-D4 polypeptide is administered to a mammal by any of a number of standard methods suitable for introducing a polypeptide into a mammal (e.g., by injection), including methods for introducing expression vectors (e.g., viral expression vectors or plasmid expression vectors). It is important that the method of administration result in a low level of polypeptide degradation so that the polypeptide retains its specific binding properties. In some embodiments, the PA-D4 polypeptide includes a detectable label (e.g., those described above). Binding of the labeled PA-D4 polypeptide can be determined in some embodiments, by detecting the label in situ, or alternatively, by isolating cells (e.g., lymphocytes) from the test animal and detecting the label ex vivo. Successful inhibition of PA-D4 polypeptide binding to the β2αA expressed on a cell surface, will be indicated by a relative decrease in the signal that is detected.

In some embodiments, a lethal dose of a combination of purified full length anthrax protective antigen polypeptide and anthrax lethal factor polypeptide, are administered to a mammal, preferably an anesthetized animal (e.g., an anesthetized rat). For example, for a rat weighing between 200-250 g, a lethal dose would be approximately 8 μg of anthrax protective antigen and approximately 40 μg of anthrax lethal factor. In other embodiments, an experimental animal (e.g., a rabbit), is challenged with a lethal dose of aerosolized B. anthracis spores, for example a 100× lethal dose 50 (LD50), where 1 Ames LD50 equals 105,000 colony-forming units), that generally results in death of the animal in approximately 3 days.

In vivo binding of PA-D4 can be inhibited by administering β2αA antibodies. In one embodiment antibodies are administered to an experimental animal at least five minutes prior to administration of the PA-D4 polypeptide. In some embodiments, an experimental animal is administered a full length anthrax protective antigen polypeptide in combination with an anthrax lethal factor toxin. Successful inhibition of anthrax protective antigen binding is indicated by prevention or delay of death of the experimental animal. In some embodiments, β2αA antibodies are administered to an experimental animal that has been or will be exposed to a pathogen that expresses an anthrax protective antigen-dependent toxin (e.g., B. anthracis). The antibodies can be administered more than once (e.g., once per day) after the experimental animal is exposed to the pathogen until death of the experimental animal by anthrax intoxication or until a successful immune response is mounted against the pathogen. Antibodies can be administered in a dose range of between about 0.04 mmole/kg of body mass to about 40 nmole/kg of body mass. Preferably, the antibodies are generated in or derived from a species that is the same as that of the experimental animal.

In other embodiments, a purified β2αA polypeptide is administered to an experimental subject to inhibit PA-D4 binding in the subject. In some embodiments, the administered β2αA polypeptide has at least one mutation that maintains the A domain in an open, high ligand affinity state. In some embodiments, an experimental subject is administered an expression vector that encodes a β2αA polypeptide. Any standard vector for in vivo expression can be used (e.g., viral vectors such as adeno-associated virus, lentivirus, adenovirus, etc). Appropriate vectors include, for example, those that result in high level expression in their expression host. In some embodiments, expression of the polypeptide encoded by an expression vector may be targeted to a specific cell type (e.g., lymphocytes). Expression can be limited to a particular cell type by using a cell-type selective promoter to drive expression of the encoded polypeptide and/or by using a vector system that has tropism for particular cell types.

### VI. Prophylactic and Therapeutic Methods for Inhibiting Anthrax Intoxication

The antibodies and polypeptides described can be used prophylactically and/or therapeutically to inhibit binding of an anthrax toxin that includes a PA-D4 in a subject. The subject in need can be a human that is either at high risk for exposure to an anthrax pathogen (e.g., B. anthracis) or an anthrax toxin, or the human one that may have already been exposed to an anthrax pathogen (e.g., B. anthracis) or an anthrax toxin.

The β2αA polypeptides (e.g., peptides that are predominantly in the open form) and the antibodies to a β2αA described herein can be incorporated into pharmaceutical compositions for use in prophylactic and therapeutic methods. Such compositions typically include the polypeptide or antibody and a pharmaceutically acceptable carrier. As used herein the language “pharmaceutically acceptable carrier” includes solvents, dispersion media, coatings, antibacterial and anti-fungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, intravenous, intradermal, subcutaneous, oral, inhalation, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethelene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some embodiments, the active components are prepared with carriers that will protect the active components against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any component used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms). Such information can be used to more accurately determine useful doses in humans. Levels in plasma may be measured, for example, by radioimmunoassay to detect the administered polypeptides or antibodies.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered at least one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

### VII. Diagnostic Assays to Detect PA-D4 Polypeptides

A competitive binding assay can be used to determine the presence and/or concentration of PA-D4 polypeptides present in a sample, e.g., a biological sample taken from a patient. In one embodiment, a series of test samples with an identical, but unknown concentration of PA-D4 polypeptide are each mixed with a detectably labeled β2αA ligand and then the mixture is contacted with an immobilized β2αA polypeptide (e.g., immobilized on beads). PA-D4 binding to labeled β2αA is then allowed to proceed. Suitable detectable labels for the β2αA ligand include any of the types of detectable label discussed above (e.g., a fluorophore, a radiolabel, etc). After binding is allowed to proceed, unbound components are washed away and the bound label is detected and/or quantified. Due to competitive binding, the amount of bound label will be inversely proportional to the concentration of PA-D4 in the unknown test sample. Examples of labeled ligands that can be used to bind to a β2αA polypeptide include, for example, a PA-D4 polypeptide, an ICAM1 polypeptide, or IC3b. PA-D4 polypeptide concentration can be quantified by determining a PA-D4 standard concentration curve. The standard concentration curve can be generated by using a range of known concentrations of PA-D4 polypeptide (e.g., spanning at least three logarithmic₁₀ units). The concentration of PA-D4 in an unknown sample can then be inferred by comparison with the standard concentration curve. The principle of the method is similar to other competitive binding methods known in the art (e.g., radioimmunoassay).

In some embodiments, a detectable label is an enzyme which can be reacted with a chromogenic substrate. Examples of suitable enzymes include horse radish peroxidase, alkaline phosphatase, β-galactosidase, luciferase, urease, glucose oxidase for which a number of chromogenic or chemiluminescent substrates and standard means for quantifying their respective reaction products are used. In some embodiments a detectable label can be a radioactive isotope, for example ¹²⁵I or ³⁵S and quantification of the detected label can be accomplished by quantifying radioactive emission using appropriate apparatus such as a gamma emission counter or a scintillation counter.

In some embodiments, an immobilized β2αA polypeptide and a β2αA ligand are labeled with a FRET donor fluorophore and a FRET acceptor fluorophore, respectively. The donor fluorophore-labeled β2αA polypeptide and the acceptor fluorophore labeled β2αA ligand are provided as a pre-formed complex and a test sample with an unknown concentration of a PA-D4 is contacted with the complex. Competitive binding of the PA-D4 to the donor fluorophore-labeled β2αA polypeptide results in a relative decrease of complexes formed between the two labeled polypeptides and thus decreased FRET. Reduced FRET can be detected as an increase in fluorescence at the donor emission wavelength. Accordingly, a standard curve for the concentration of a PA-D4 can be established as described above, using a fluorescence assay to measure donor fluorophore emission as an endpoint

### VIII. Testing Compounds for Inhibition of PA-D4 to β2αA Binding

In some embodiments, test compounds are screened for their ability that inhibit PA-D4 to β2αA binding. Screening methods for binding inhibitors include conducting one of the PA-D4 to β2αA bindings assays described herein, in the presence or absence of a test compound. The methods can be performed in vitro, e.g., in a cell free system, or ex vivo.

The compounds to be tested can be, e.g., proteins, peptides, peptidomimetics, peptoids, and small molecules. The test compounds can be obtained from combinatorial libraries including: biological libraries; peptoid libraries; spatially addressable parallel solid phase or solution phase libraries; synthetic library methods requiring deconvolution; “one-bead one-compound” libraries.

Libraries of compounds may be presented in solution (e.g., Houghten 1992 Biotechniques 13:412-421), or on beads (Lam 1991 Nature 354:82-84), chips (Fodor 1993 Nature 364:555-556), bacteria (Ladner U.S. Pat. No. 5,223,409), spores (Ladner U.S. Pat. No. 5,223,409), plasmids (Cull et al. 1992 Proc Natl Acad Sci USA 89:1865-1869) or on phage (Scott and Smith 1990 Science 249:386-390); Devlin 1990 Science 249:404-406; Cwirla et al. 1990 Proc. Natl. Acad. Sci. 87:6378-6382; Felici 1991 J. Mol. Biol. 222:301-310; Ladner, supra.).

In vitro testing of a compound for its ability to inhibit PA-D4 to β2αA binding can be done using any of the methods described above, for determining binding of purified polypeptides or binding of a purified polypeptide to a polypeptide expressed on the surface of a cell, except that binding of the polypeptides is carried out in the presence of a test compound, preferably using various concentrations of the test compound.

In other embodiments, a β2αA polypeptide can be used as a “bait protein” and a PA-D4 polypeptide is used as a “prey protein,” in a binding assay that utilizes a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; Hubsman et al. (2001) Nuc. Acids Res. February 15; 29(4):E18; and Brent WO94/10300).

A two-hybrid assay can be carried out using a β2αA domain as the bait protein. Briefly, the variant A domain is fused to the LexA DNA binding domain and used as bait. The prey is a PA-D4 polypeptide library cloned into the active site loop of TrxA expression as a fusion protein with an N-terminal nuclear localization signal, a LexA activation domain, and an epitope tag (Colas et al. 1996 Nature 380:548; and Gyuris et al. Cell 1993 75:791). Yeast cells are transformed with bait and prey genes. When the PA-D4 fusion protein binds to the β2αA fusion protein, the LexA activation domain is brought into proximity with the LexA DNA binding domain and expression of reporter genes or selectable marker genes having an appropriately positioned LexA binding site increases. In some embodiments, a compound can be tested for its ability to disrupt the interaction of the β2αA bait and PA-D4 prey fusion proteins. Disruption of bait-prey interactions is reflected by a decrease in the expression of a LexA-dependent reporter gene and/or a selectable marker gene. Compounds that decrease bait-prey interactions are likely candidates for disrupting β2αA-PA-D4 interactions. Suitable reporter genes include fluorescent proteins (e.g., EGFP), enzymes (e.g., luciferase, β-galactosidase, alkaline phosphatase, etc.) Suitable selectable marker genes include, for example, the yeast LEU2 gene.

### IX. PA-D4 and β2αA Polypeptides

PA-D4 polypeptides, include any polypeptide that includes an amino acid sequence that is at least 90% identical (e.g., 92, 94, 96, 98, 99%, or 100% identical) to SEQ ID NO:1 and can bind to a polypeptide that includes a β2αA can be used in the various methods described herein.

Polypeptides that have a β2αA (β2A polypeptides) are those polypeptides that include an amino acid sequence that is at least 90% identical (e.g., 92%, 94%, 96%, 98%, 99%, or 100% identical) to that of any of the β2αAs listed as SEQ IDs:2-10.

β2αAs can be in a structurally open state which is competent to bind a ligand with high affinity or a closed state that does not bind a ligand with high affinity or can switch between these two forms in solution. Detailed structural characteristics of β2αAs in the open and closed states can be found in U.S. patent application Ser. No. 09/805,354 of Arnaout et al. and Xiong et al., J. Biol. Chem., 275(49):38762-38767, (2000), incorporated herein by reference.

An α subunit A domain that is part of a β2 integrin α subunit expressed by a cell can adopt an open state via a cellular signal transduction process known as “inside-out activation.” Inside-out activation of a β2αA can occur spontaneously, (i.e., without an experimental manipulation for that purpose), or inside-out activation can be induced by presenting certain stimuli to the cell. Non-limiting examples of such stimuli include exposing the cell to phorbol myristate acetate or Mn²⁺.

It is also possible to generate a β2αA that stably adopts the open state, by introducing one or more amino acid substitutions. Structural data indicate that mutation of an invariant isoleucine residue in β2αAs, corresponding to Ile 332, located in the A domain of the β2 integrin α subunit, CD11b, can generate a stable open form of the αA domain, that has high ligand affinity. Suitable mutations of Ile 332 include Ile332Gly and Ile332Ala, for example. Table 1, lists the position of the invariant isoleucine residue for a series of human a integrin subunits and lists the range of amino acids for the corresponding A domain and refers to the corresponding SEQ ID NO (e.g., C144-A344 for human CD11b is SEQ ID NO:2). Note that the amino acid residue numbering listed in Table 1 corresponds to that of integrin precursor polypeptides which include a 16 amino acid signal sequence not present in the mature polypeptide. For example, in the case of CD11b, isoleucine 332 of the precursor polypeptide (listed in Table 1) corresponds to isoleucine 316 of the mature polypeptide (e.g., as described in Example 1). In addition, all sequence figures and listings refer to integrin precursor sequences.

TABLE 1

| | | Invariant | | | |
|---|---|---|---|---|---|
| | GenBank | Ile Position | | SEQ | |
| Integrin α | Accession | in whole | | ID | |
| Subunit | No. | integrin | A domain | NO. | FIG. |
| Human CD11b | RWHU1B | Residue 332 | C144-A334 | 2 | 6 |
| Human CD11c | RWHU1C | Residue 333 | C145-A335 | 3 | 7 |
| Human CD11d | AAB38547 | Residue 332 | C144-A334 | 4 | 8 |
| Human CD11a | AAC31672 | Residue 331 | C150-V333 | 5 | 9 |
| Human Alpha | P56199 | Residue 331 | C139-A333 | 6 | 10 |
| 1 (CD49a) | | | | | |
| Human Alpha | NP_002194 | Residue 361 | C169-S363 | 7 | 11 |
| 2 (CD49b) | | | | | |
| Human Alpha | O75578 | Residue 249 | C57-G251 | 8 | 12 |
| 10 | | | | | |
| Human Alpha | NP_036343 | Residue 349 | C159-S351 | 9 | 13 |
| 11 | | | | | |
| Human Alpha | A53213 | Residue 385 | E196-S387 | 10 | 14 |
| E | | | | | |

The Ile can also be substituted by an amino acid other than Gly or Ala.

Stable, open (high affinity) forms of β2αAs, can also be created by substituting the Glu at 330 of CD11b with another amino acid, e.g., Gly or Ala. A corresponding change can be made in any of the other polypeptides listed in Table 1.

Stable, open (high affinity) forms of β2αAs, can also be created by substituting certain amino acid residues with cysteine residues, so as to create a disulfide bridge that stabilizes the open form of the β2αA. Table 2 indicates the residues that should be changed to cysteines in order to prepare stable open forms of the indicated β2αAs.

TABLE 2

| Integrin α Subunit | Mutations for Stable Open Form |
|---|---|
| Human CD11b | A320→C |
| | F313→C |
| Human CD11c | A321→C |
| | F314→C |
| Human CD11d | A320→C |
| | F313→C |
| Human CD11a | K319→C |
| | F312→C |
| Human Alpha 1 | A319→C |
| (CD49a) | F312→C |
| Human Alpha 2 | A349→C |
| (CD49b) | F342→C |
| Human Alpha 10 | A337→C |
| | F330→C |
| Human Alpha 11 | A337→C |
| | F330→C |
| Human Alpha E | A373→C |
| | V366→C |

Other β2αA polypeptides that can be used in the methods described in this application are variant integrin α subunits in which the invariant Ile, referred to in Table 1, has been deleted. Also included are polypeptides that have the entire A domain of an integrin a except for the invariant Ile. For example, amino acids 144-332 of CD11b, amino acids 145-332 of CD 11c, amino acids 144-331 of CD11d, amino acids 150-330 of CD 11a, but does not include the remainder of the integrin α subunit. Other useful polypeptides are those comprising the A domain of an integrin α subunit up to but not including the invariant Ile and further lacking the 5 amino acids following the invariant Ile (e.g., amino acids 144-331, but not 332-336 of CD11b; amino acids 145-332, but not 333-337 of CD11c; amino acids 144-331, but not 332-336 of CD11d; amino acids 150-330, but not 331-335 of CD11a; amino acids 139-330, but not 331-335 of human alpha 1; amino acids 169-360, but not 361-335 of human alpha 2; amino acids 57-248, but not 249-253 of human alpha 10; amino acids 159-348, but not 349-353 of human alpha 11; or amino acids 196-384, but not 385-389 of human alpha E). Also useful in the methods described in the application, are heterodimers, that include a β2αA and β2βA-like domain (e.g., a β2 integrin ectodomain or a β2 integrin receptor).

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

A particularly preferred set of parameters (and the one that should be used if the practitioner is uncertain about what parameters should be applied to determine if a molecule is within a homology limitation of the invention) are a Blossum 62 scoring matrix with a gap penalty of 12, a gap extend penalty of 4, and a frameshift gap penalty of 5.

The polypeptides described above can be part of a fusion protein which includes all or a portion of a second polypeptide that is not a β2 integrin A domain or an anthrax protective antigen domain D4 domain. This second polypeptide can be fused to the C-terminus or the N-terminus of the variant integrin polypeptide. All or part of a third peptide may also be present. Thus, a variant integrin polypeptide can be fused to, e.g., GST, an immunoglobulin constant region, a heterologous signal sequence, a reporter polypeptide (e.g., a GFP variant, a luciferase, an alkaline phosphatase), a short amino acid sequence tag.

### X. β2αA and β2βA-Like Domain Antibodies

In preferred embodiments, the anti integrin A or A-like domain antibodies described below, inhibit the binding of a polypeptide that includes a PA-D4 polypeptide to a polypeptide that includes a β2αA. The ability of an antibody to inhibit PA-D4 to β2αA binding can be tested by the methods described herein or by similar methods.

β2αA polypeptides or β2 integrin β subunit A like domain polypeptides can be used as immunogens or antigens to generate antibodies, including antibodies that bind specifically to an open form of a β2 integrin α subunit A domain.

In one embodiment the disclosed methods feature the use of antibodies directed against open form (high ligand affinity) variants of β2αA polypeptides. Such antibodies bind to an open form of a β2αA with greater affinity than they bind to a closed form of a β2αA. Open form-specific antibodies also recognize, native β2 integrin receptors expressed on the cell surface, that have undergone “inside-out activation”, a signal transduction event which renders the receptor competent to bind ligands.

Disclosed methods feature the of use of antibodies to variant β2αAs that have amino acid substitutions according to Tables 1 and/or 2. The polypeptide used to generate the open form-specific antibodies can include part or all of the indicated β2αA, e.g., 10, 20, 30, 40, 50, 60, 70, 80, 90, 100, 110, 120, 130, 140, 150, 160, 170, 180, 190, or 200 contiguous amino acids of the A domain that includes a mutation of the Ile residues listed in Table 1 and alternatively can include a double cysteine substitution corresponding to the positions listed in Table 2. Open form-specific antibodies (e.g., mAb KIM127) are useful for inhibiting ligand binding to a β2 integrin receptor and are particularly useful for blocking PA-D4 binding to an open form of a β2αA (e.g., an A domain that has undergone inside-out activation, or a stable open form variant of β2αA including one that has a mutation according to Tables 1 or 2.

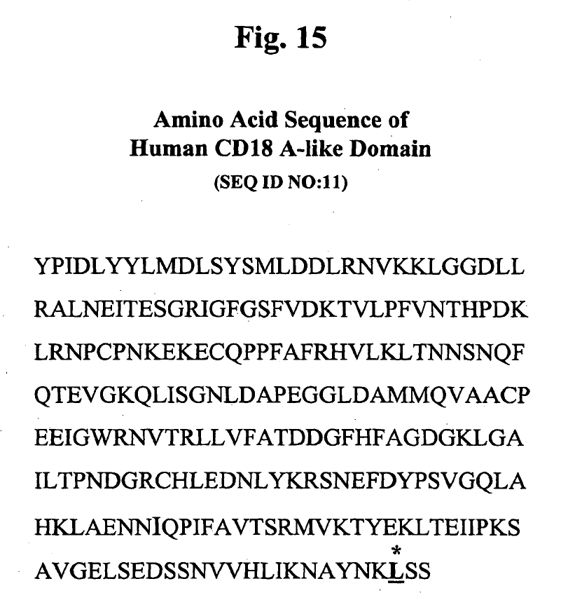

Also disclosed is the use of antibodies that can bind with high affinity to integrin β2 integrin β subunit A like domain polypeptides and in particular to polypeptides derived from CD18, including polypeptides that include an amino acid sequence that is at least 90% identical (e.g., 92%, 94%, 96%, 98%, 99%, or 100% identical) to that of SEQ ID:11 (FIG. 15). Appropriate β2β A-like domain polypeptides used to generate antibodies to β2β A-like domains can include part or all of the indicated β2β A-like domain, e.g., 10, 20, 30, 40, 50, 60, 70, 80, 90, 100, 110, 120, 130, 140, 150, 160, 170, 180, 190, or 200 contiguous amino acids of the A-like domain.

In some embodiments antibodies are specific for an open form of a β2β A-like domain. Given the sequence similarity among the A-like domains of integrin β subunits, deletion or substitution of the Ile in a selected integrin, subunit that corresponds to Ile332 of CD11b should result in the creation of a variant integrin β subunit that is more active (i.e., in solution has a greater proportion of ligand binding form polypeptides) than the wild-type form of the subunit. Replacing Ile 318 with Ala or Gly or some other suitable amino acid should create a stable open form of the CD 18 integrin polypeptide.

The antibodies can be generated using standard methods and include antibodies purified in vitro, for example through screening of large phage antibody libraries for binding to a β2 integrin A domain polypeptide.

The term “antibody” refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin. The antibody can be a polyclonal, monoclonal, recombinant, e.g., a chimeric or humanized, fully human, non-human, e.g., murine, or single chain antibody. The antibody can be artificially evolved to increase its ligand affinity, for example by RT-PCR cloning of the antibody variable region gene sequences, generation of variable region phage display libraries using error prone PCR Panning of such variable region phage libraries can then be performed using the ligand of interest to select phage clones with high affinity, for example as was done in Maynard et al., Nature Biotechnol., 20(6):597-601, (2000), which is incorporated herein by reference.

Chimeric, humanized, but most preferably, completely human antibodies are desirable for applications which include repeated administration, e.g., prophylactic treatment, therapeutic treatment (and some diagnostic applications) of human patients.

The antibodies to be used in the disclosed methods can be a single chain antibody which can be optionally dimerized or multimerized to generate multivalent antibodies. The antibody can be designed to have little or no ability to bind to an Fc receptor.

Detection of bound antibody can be facilitated by coupling (i.e., physically linking) the antibody to a detectable label (i.e., antibody labeling). Examples of detectable labels include various enzymes, prosthetic groups, fluorescent materials, luminescent materials, bioluminescent materials, radioactive materials, magnetic resonance imaging contrast materials (e.g., horseradish peroxidase, alkaline phosphatase, β-galactosidase, acetylcholinesterase, streptavidin/biotin and avidin/biotin, umbelliferone, fluorescein, fluorescein isothiocyanate, rhodamine, dichlorotriazinylamine fluorescein, dansyl chloride or phycoerythrin, luminol, luciferase, luciferin, enhanced green fluorescent protein, ¹²⁵I, ¹³¹I, ³⁵S ³H, and gadolinium).

### EXAMPLES

### Example 1

### Interaction of the β2αA Domain of Integrin CD11b/CD18 with the PA-D4

The β2αA is the ligand binding domain of integrin CD11b/CD18. This domain has been proposed to function as “ligand relay” to the β2 integrin β subunit A-like domain of CD18 though an acid residue at its C-terminal. The recombinant β2αA stays in “closed” or low affinity status. The “open” or high affinity status can be obtained by either deletion of one turn helix at the C-terminal site of the α7 helix, or a mutation I316G in this helix. Note that the residue numbering in these and other examples refers to that of the mature polypeptide, which lacks a 16 amino acid signal peptide present in the precursor polypeptide (i.e., I316 corresponds to I332 of the precursor polypeptide). PA-D4 is the major binding domain of anthrax protective antigen. Both “closed” and “open” β2αAs were used to test their binding to PA-D4, using surface plasmon resonance. In the crystal structure of the I316G mutant, residue E314 was found to coordinate to the metal ion-dependent adhesion site (MIDAS), Xiong et al., J. Biol. Chem., 275:38762-38767, (2000), raising the possibility that the open form may be induced by the αA domain itself. In order to exclude this possibility, a second site mutation (E314A) was introduced and the resulting (E314A/I316G) double mutant was also used in a binding assay.

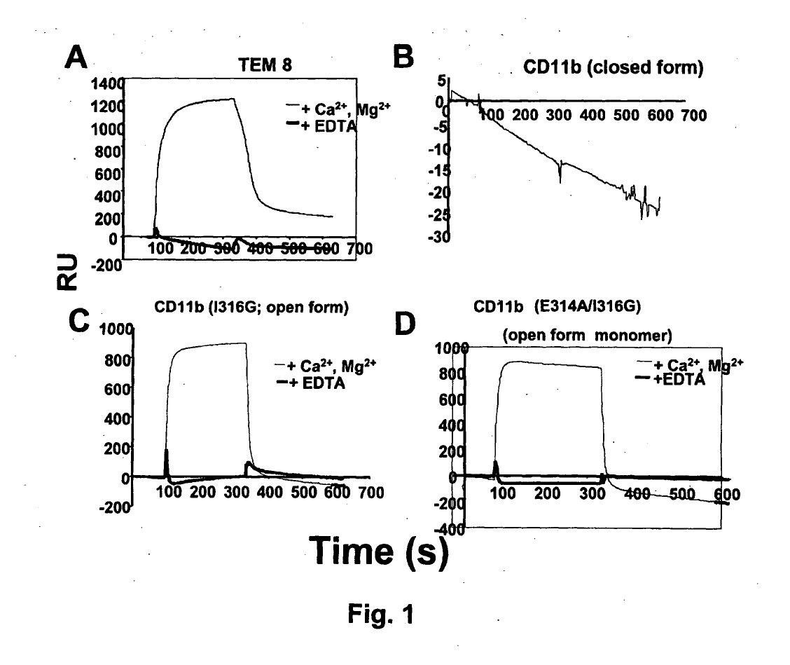

In a positive control experiment, PA-D4 binds to the TEM 8A domain as shown in FIG. 1A in the presence of 1 mM Ca2⁺ and Mg2⁺ (thin line curve). As expected, the binding was totally abolished in the presence of 5 mM EDTA (thick line curve). Both “closed” and “open” β2αAs were used to test their binding to PA-D4, using surface plasmon resonance.

Both the I316G and E314A/I316G mutants A domains, which are “open”, high affinity forms of the αA domain of CD11b/CD18, bound PA-D4 with high affinity in the presence of 1 mM Ca²⁺ and Mg²⁺ (FIG. 1C, D, thin line curves). The binding was abolished in the presence of 5 mM EDTA (FIG. 1 C, D, thick line curves), strongly indicating that the binding of the PA-D4 to the integrin α A domain is specific, since the binding of β2 integrin ligands is known to be cation-dependent. The Kd for this interaction was about 20 nM, which is comparable to that of the mutant with its physiological ligand.

No binding of the closed, low affinity β2αA of integrin CD11b to PA-D4 was detected (FIG. 1B), which is also the case for the binding of known physiological ligands to the closed form of CD11b/CD18 and to closed form β2.integrins generally.

### Example 2

### Inhibition of Anthrax PA Binding to Purified CD11b/CD18 Ectodomain by a Monoclonal Antibody to the CD11b A Domain

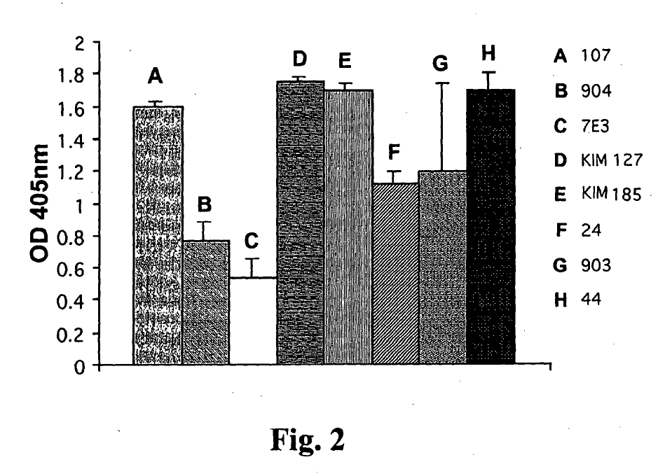

A number of monoclonal antibodies were tested for their ability to bind to a purified open form of the CD11b/CD18 ectodomain, by a solid phase assay as shown in FIG. 2. In brief, 100 μl of 50 ng/ml of CD 11b/CD18 in Tris-buffered saline (TBS) were added in triplicate to a 48-well plate and incubated overnight at 4° C. The plate was then washed with T-TBS (TBS with 0.1% Tween) and blocked with 1% bovine serum albumin (BSA) in T-TBS. 50 μl of 10 μg/ml of various antibodies (in TTBS+1% BSA) were added to the wells and incubated for 1 hour at room temperature. The unbound antibodies were washed away with TTBS. Alkaline phosphatase-conjugated secondary antibodies (diluted at 1:30,000) were then added to each well and incubated for 1 hour at room temperature. The unbound antibody was washed away with TIBS. 50 μl of 0.1 M alkaline phosphatase substrate p-nitrophenyl-phosphate (Sigma) in 20 mM sodium bicarbonate buffer pH 8.5, was added and incubated for at least fifteen minutes at 37° C. Afterwards the plate was placed in a microplate reader and the optical absorbance at 405 nm (OD 405) was determined. FIG. 2 shows a panel of monoclonal antibodies (labeled A-H on the bar graphs) that bound to CD11b/CD18 ectodomain.

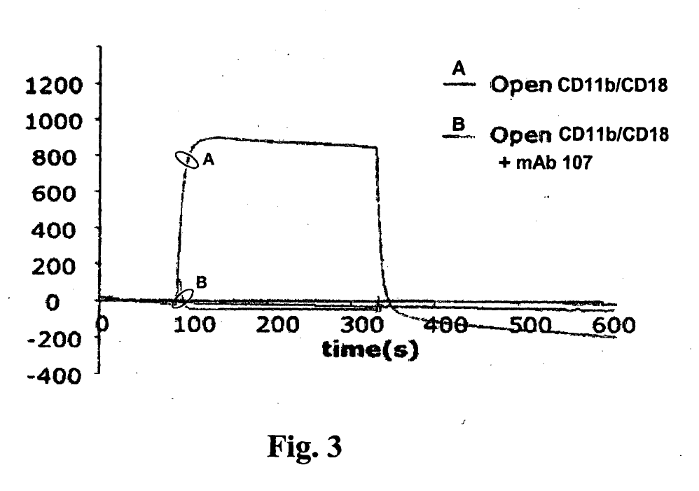

The ability of monoclonal antibody 107 (FIG. 2, bar A) to inhibit the binding of anthrax PA to purified CD11b/CD18 ectodomain was tested by a surface plasmon resonance binding assay. As shown in FIG. 3, the high affinity binding of PA to open form (high ligand affinity) CD11b/CD18 (curve labeled A) is abolished in the presence of mAb 107 at 10 μg/ml, thus demonstrating that the monoclonal antibody is an effective inhibitor of PA binding to CD11b/CD18.

### Example 3

### Inhibition of Anthrax PA Binding to Cellular β2 Integrin Receptors and Decreased PA-Dependent Toxicity, by Competitive Binding of Von Willebrand Type A Domain Polypeptides to PA-D4

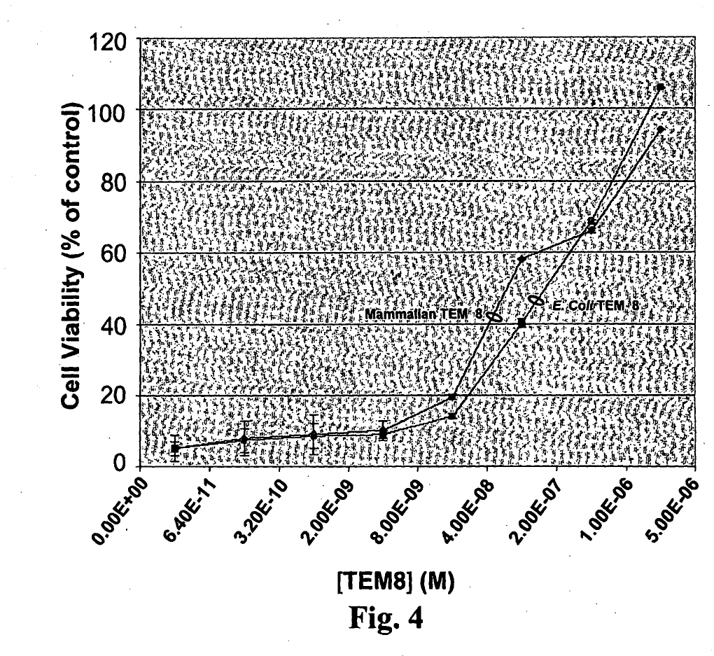

Cells (approximately 15,000) that expressed integrins were seeded on each well of a 96-well plate. The following day, they were exposed to 10⁻¹⁰M full length anthrax protective antigen polypeptide and 10⁻¹⁰M LFn-DTA, a recombinant protein with the N-terminal PA-binding region of anthrax lethal factor fused to the diphtheria toxin catalytic A chain, in the presence or absence of an A-like domain from TEM8. Cells were incubated with toxin/sTEM8 for 24 hours, then viability was measured using commercially available assay (WST-1 assay; Roche Molecular Biochemicals). All points were performed in triplicate. Viability was determined for each condition and compared against cells that received PA only. As indicated in FIG. 4, the Von Willebrand type A domain of TEM8 served as an effective competitive inhibitor of PA binding to β2 integrin receptors, inhibiting PA-dependent toxicity in a concentration dependent manner.

### Other Embodiments

A number of embodiments of the invention have been described. Nevertheless, it will be understood that various modifications may be made without departing from the spirit and scope of the invention. Accordingly, other embodiments are within the scope of the following claims.

1. A method of inhibiting the binding of a polypeptide comprising a fragment of a PA-D4 to a polypeptide comprising a β2 integrin α subunit A domain, the method comprising contacting the β2 integrin α subunit A domain with an antibody that binds to the β2 integrin α subunit A domain.

2. (canceled)

3. The method of claim 1 wherein the β2 integrin α subunit A domain is at least 90% identical to any of SEQ ID NOs:1-10.

4. The method of claim 1, wherein the β2 integrin α subunit A domain is the CD11b A domain.

5-7. (canceled)

8. The method of claim 1, wherein the antibody can compete for binding to an epitope recognized by an antibody selected from the group consisting of mAb107, KIM127, 904, CBR, TS1/18, IB4, 24, LM2-1, 60.3, OKM9, OKM1, M1/70, M1/1, M1/2, M1/4, M1/9, M1/10, M1/13, M1/15, M1/16, M1/17, M1/18, M1/20, M1/21, M1/22, M1/23, M1/24, M1/25, M1/26, M1/27, M1/28, M1/29, M1/30, M1/31, M1/32, M1/33, and M1/34.

9-11. (canceled)

12. The method of claim 1, wherein the polypeptide comprising the β2 integrin α subunit A domain is a purified polypeptide.

13-21. (canceled)

22. A method of inhibiting the binding of a polypeptide comprising a fragment of PA-D4 to a first polypeptide comprising a β2 integrin α subunit A domain, the method comprising contacting the polypeptide comprising a fragment of PA-D4 with a second polypeptide comprising a β2 integrin α subunit A domain.

23. (canceled)

24. The method of claim 22, wherein the amino acid sequence of the β2 integrin α subunit A domain of the first polypeptide comprising a β2 integrin α subunit A domain is at least 90% identical to any of SEQ ID NOs:1-10.

25-42. (canceled)

43. A method of identifying a compound that inhibits the binding of a polypeptide comprising a fragment of a PA-D4 to a polypeptide comprising a β2 integrin α subunit A domain, the method comprising:
a. providing the polypeptide comprising a β2 integrin α subunit A domain;
b. contacting the polypeptide comprising a β2 integrin α subunit A domain with the polypeptide comprising PA-D4 and also with a test compound; and
c. determining whether binding of the polypeptide comprising a fragment of a PA-D4 to the polypeptide comprising a β2 integrin α subunit A domain is decreased in the presence of the test compound relative to binding of the polypeptide comprising a fragment of PA-D4 to the polypeptide comprising a β2 integrin α subunit A domain in the absence of the test compound, a decrease in the binding being an indication that the test compound inhibits the binding of the polypeptide comprising a fragment of PA-D4 to the polypeptide comprising a β2 integrin α subunit A domain.

44. (canceled)

45. The method of claim 43, wherein the amino acid sequence of the β2 integrin α subunit A domain is at least 90% identical to that of any of SEQ ID NOs:1-10.

46-50. (canceled)

51. The method of claim 43, wherein the polypeptide comprising a β2 integrin α subunit A domain is a purified polypeptide.

52. The method of claim 43, wherein the polypeptide comprising a fragment of PA-D4 is a purified polypeptide.

53. The method of claim 43, wherein the polypeptide comprising a β2 integrin α subunit A domain is expressed by a cell.

54. The method of claim 43, wherein the polypeptide comprising a fragment of PA-D4 is expressed by a cell.

55-98. (canceled)


SEQUENCE LISTING (11 sequences)

SEQ ID NO: 1 - protein, 140 aa, Bacillus anthracis
FHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 191 aa, Homo sapiens
CPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFA

SEQ ID NO: 3 - protein, 191 aa, Homo sapiens
CPRQEQDIVFLIDGSGSISSRNFATMMNFVRAVISQFQRPSTQFSLMQFSNKFQTHFTFEEFRRTSNPLSLLASVHQLQGFTYTATAIQNVVHRLFHASYGARRDATKILIVITDGKKEGDSLDYKDVIPMADAAGIIRYAIGVGLAFQNRNSWKELNDIASKPSQEHIFKVEDFDALKDIQNQLKEKIFA

SEQ ID NO: 4 - protein, 191 aa, Homo sapiens
CPHQEMDIVFLIDGSGSIDQNDFNQMKGFVQAVMGQFEGTDTLFALMQYSNLLKIHFTFTQFRTSPSQQSLVDPIVQLKGLTFTATGILTVVTQLFHHKNGARKSAKKILIVITDGQKYKDPLEYSDVIPQAEKAGIIRYAIGVGHAFQGPTARQELNTISSAPPQDHVFKVDNFAALGSIQKQLQEKIYA

SEQ ID NO: 5 - protein, 184 aa, Homo sapiens
CIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYV

SEQ ID NO: 6 - protein, 193 aa, Homo sapiens
CGNQYYTTGVCSDISPDFQLSASFSPATQPCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKA

SEQ ID NO: 7 - protein, 195 aa, Homo sapiens
CPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFS

SEQ ID NO: 8 - protein, 195 aa, Homo sapiens
CPTYMDVVIVLDGSNSIYPWSEVQTFLRRLVGKLFIDPEQIQVGLVQYGESPVHEWSLGDFRTKEEVVRAAKNLSRREGRETKTAQAIMVACTEGFSQSHGGRPEAARLLVVVTDGESHDGEELPAALKACEAGRVTRYGIAVLGHYLRRQRDPSSFLREIRTIASDPDERFFFNVTDEAALTDIVDALGDRIFG

SEQ ID NO: 9 - protein, 193 aa, Homo sapiens
CQTYMDIVIVLDGSNSIYPWVEVQHFLINILKKFYIGPGQIQVGVVQYGEDVVHEFHLNDYRSVKDVVEAASHIEQRGGTETRTAFGIEFARSEAFQKGGRKGAKKVMIVITDGESHDSPDLEKVIQQSERDNVTRYAVAVLGYYNRRGINPETFLNEIKYIASDPDDKHFFNVTDEAALKDIVDALGDRIFS

SEQ ID NO: 10 - protein, 193 aa, Homo sapiens
EEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIIS

SEQ ID NO: 11 - protein, 241 aa, Homo sapiens
YPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSS